(19) United States
(12) Patent Application Publication
(10) Pub. No.: US 2004/0037855 A1
(43) Pub. Date: Feb. 26, 2004

(54) Deer scent composition

(76) Inventor: Larry Keith Jones, Brusly, LA (US)

Correspondence Address: David L. Ray, Attorney for Applicant, Ste. 205, 2051 Silverside Drive, Baton Rouge, LA, 70808, US

(21) Appl. No.: 10/224,148

(22) Filed: Aug. 20, 2002

(51) Int. Cl. A61K 7/46

(52) U.S. Cl. 424/401; 512/1

(57) ABSTRACT

A scent composition for masking the odor of a hunter to prevent deer from detecting the scent of a hunter, and as a lure to attract deer to a hunter. The scent composition of the invention includes deer urine and deer fecal matter.

### BACKGROUND OF THE INVENTION

1. Field of the Invention

The present invention relates to scent compositions for masking the odor of a hunter to prevent deer from detecting the scent of the hunter, and to scent compositions for attracting deer to a hunter. In particular the present invention relates to scent compositions containing urine of an animal which is placed in the vicinity of a hunter to mask the scent of the hunter and to attract the animal to a desired area.

2. Description of the Related Art

As is known in the art, buck deer in the rut commonly mark there territory in a forest with scrapes where they scratch or scrape leaves and soil with their hooves from a small area in the forest. They then urinate on the scrape to mark their territory. Urination occurs on their legs which causes scent from their tarsal glands, femoral glands, and interdigital glands to be deposited on the scrape. Doe deer are visually attracted to the scrape and are also attracted to the scrape by the scent left by the buck deer which made the scrape

Hunters, game observers, and wildlife photographers have long used various lures and scents in the vicinity of scraps and other areas to mask their odors and to attract desired animals to selected locations. Many such products are known in the art. Exemplary of the patents of the related art are the following: U.S. Pat. Nos. 4,213,875; 4,472,377; 4,944,940; 5,041,421; 5,303,496; 5,565,111; 5,896,692; 5,901,490; 5,916,552; 6,129,892; 6,149,901; 6,199,311 Bi and Japanese Patent 10094592.

### BRIEF SUMMARY OF THE INVENTION

In accordance with the present invention there is provided a scent composition for masking the odor of a hunter to prevent deer from detecting the scent of a hunter, and as a lure to attract deer to a hunter. The scent composition of the invention includes deer urine and deer fecal matter.

### DETAILED DESCRIPTION OF THE INVENTION

The composition of the present invention for masking the odor of a hunter and for attracting deer to the hunter includes deer fecal matter (deer feces) and urine. Preferably, the scent composition of the invention is mixed with decomposed forest material. More preferably, a preservative such as salt, preferably sodium chloride, and/or granular phosphate is also mixed with the scent composition of the invention. Even more preferably, the scent composition of the present invention includes skin particles of deer, deer hair and scent of the interdigital gland of the deer. Most preferably, sand such as river sand and the like is also mixed with the scent composition of the invention.

Urine and fecal matter from the particular species of deer or other animal to be hunted is collected from animals which are herded into a pen, stall or other holding facility having soil gathered from a forest composed of decomposed forest material. The decomposed forest material can include decomposed leaves, branches and common soils.

The animals are held and fed in the pen or stall for a time sufficient for the animals to defecate and urinate into the soil in the stall, usually two weeks or fourteen days. The decomposed forest material in the pen or stall containing the animal urine, feces, skin particles, hair, and scent from the interdigital glands is then removed from the pen or stall and sand and salt or other preservatives are added thereto and stored in drums or other containers. The scent composition is then preferably placed in small plastic bags, preferably about one pound in size, and sealed to prevent the scent composition and odors therein from escaping.

Preferably the preservative comprises from about 5% to about 10% by weight of the total weight of the scent composition of the invention.

Preferably the sand included in the scent composition comprises from about 60% to about 70% by weight of the total weight of the scent composition of the invention.

### EXAMPLE

Several whitetail deer including bucks and does are placed in a stall on areas of the ground having decomposed forest material therein. The deer are left in the stall for about 14 days and defecate and urinate on the decomposed forest material in the stall. The decomposed forest material in the stall is removed and 5% by weight of sodium chloride is added thereto and the decomposed forest material is placed in one pound plastic bags and the bags are sealed to prevent odor and scent from escaping therefrom.

A hunter or other wildlife observer or photographer then transports a bag of the scent composition of the invention to a desired area containing deer or other animals to be hunted and spreads the scent composition upon the ground where the deer or other animals travel. Preferably, the scent composition of the invention is used in or around existing deer scrapes or wallows. Also the scent composition of the invention may be spread around animal bait piles to attract the desired animal, such as a deer.

If desired the scent composition of the invention may be used alone or in conjunction with other commercially available deer urine, deer scent, or deer lures.

Although the preferred embodiments of the invention have been described in detail above, it should be understood that the invention is in no sense limited thereby, and its scope is to be determined by that of the following claims:

What is claimed is:

1. A scent composition for masking the odor of a hunter to prevent deer from detecting the scent of a hunter and to attract deer to a hunter, said scent composition comprising:
a. deer urine, and
b. deer feces.

2. The scent composition of claim 1 wherein said scent composition includes decomposed forest material.

3. The scent composition of claim 2 wherein said scent composition includes a preservative.

4. The scent composition of claim 3 wherein said preservative is salt.

5. The scent composition of claim 4 wherein said salt is sodium chloride.

6. The scent composition of claim 5 wherein sand is added to said scent composition.
